(19) United States
(12) Patent Application Publication
(10) Pub. No.: US 2022/0322938 A1
(43) Pub. Date: Oct. 13, 2022

(54) SYSTEM AND METHOD FOR DETERMINING INTRA-OCULAR PRESSURE

(71) Applicant: Ophthalmic Sciences Ltd, Tel-Aviv (IL)

(72) Inventors: Noam HADAS, Tel-Aviv (IL); Gabriel DAN, Tel-Aviv (IL)

(73) Assignee: Ophthalmic Sciences Ltd, Tel-Aviv (IL)

(21) Appl. No.: 17/638,240

(22) PCT Filed: Sep. 7, 2020
(86) PCT No.: PCT/IB2020/058314

§ 371 Date: Feb. 25, 2022

Related U.S. Application Data
(60) Provisional application No. 62/900,824, filed on Sep. 16, 2019.

(51) Int. Cl. A61B 3/16; A61B 3/14; A61B 8/10; A61B 3/12; A61B 5/00

(52) U.S. Cl. CPC A61B 3/16; A61B 3/14; A61B 8/10; A61B 3/1216; A61B 5/6803

(57) ABSTRACT

A system for determining an lop of a subject includes a pressurizing device for applying pressure of varying magnitude over an external surface of an eye of the subject; a monitoring device for monitoring internal vasculature of the eye and vasculature on or around the eye; and a processing unit for correlating a first pressure or pressure range with pulsation or collapse of the internal vasculature of the eye and a second pressure or pressure range with pulsation or collapse of the vasculature on or around the eye to thereby derive the IOP of the subject.

### FIELD OF INVENTION

This invention relates in general to systems for determining an intra-ocular pressure. Embodiments of the present invention relate to portable tonometers which can be utilized in non-clinical settings by subjects themselves or by operators.

### BACKGROUND OF INVENTION

Measurement of intra-ocular pressure (IOP) is an important procedure in diagnosing various diseases and abnormalities of the eye as well as monitoring status of ophthalmic therapies and procedures.

IOP is measured by a device called tonometer. Traditional stationary tonometers are very bulky and are consigned to medical offices, require special training and are limited to testing IPO when the patient is in vertical position,

Some tonometers require eye contact and thus are more complicated and may require using disposable sterile parts.

Measurement of IOP in different subject's positions sometimes results in differing readings, so some medical practitioners suggest measuring it at several different positions.

Uncooperative subjects such as small children have to be sedated in order to perform IOP measurement. in case of the elderly or in an emergency room setting there's also a need to measure IOP when the subject is prone.

There is a need to provide an improved tonometer.

### SUMMARY OF THE INVENTION

There may be provided a portable tonometer which can be easily carried by or to the subject.

There may be provided a tonometer which is easy to use, so it can be operated by subject himself, in a home environment rather than by a medical specialist in a medical office.

There may be provided a tonometer that can be used with pediatric subjects with minimum preparation of the subject or operator training.

There may be provided a tonometer which can be used in any position.

There may be provided a tonometer which may cause minimal discomfort to the subject during the measurement.

There may be provided a tonometer that is relatively inexpensive.

There may be provided a tonometer which can store and transfer IOP readings to remote devices and locations.

There may be provided a tonometer that is accurate and does not require extensive training prior to operation.

There may be provided a tonometer that may be configured to measure the IOP by sampling different parts of the eye in an iterative manner—until similar results are measured at multiple sites. This provides a reliable tonometer that is more tolerable to errors in positioning than a single sampling point, and is tolerable to errors in a selection of a single sampling point due to anatomical variations between patients—such as corneal thickness.

There may be provided a tonometer that may not require any aiming, or placing a device in a specific spatial relationship with the eye.

There may be provided a tonometer that may automatically measure IOP of both eyes at the same time.

There may be provided a tonometer that may also provide readings of central ocular artery blood pressure, and other pressure values in various blood vessels or compartments which are beneficial for diagnostic or follow-up purposes.

There may be provided a tonometer that may not require any special alignment with the subject's eye, and may merely require wearing of an eye mask that may be similar to a diver's mask. This enables operation of the tonometer by subjects themselves, or in case of pediatric subjects, greatly simplifying the procedure, so that even an inexperienced operator, such as child's parent can easily measure subject's top.

There may be provided a tonometer that may be operated fully automatically, further simplifying its operation.

There may be provided a tonometer that may not require any eye contact, and may not require disposable parts in contact with the eye, and thus the tonometer can be easily used with multiple patients, and operational costs are very low.

There may be provided a tonometer that may be used to perform IOP measurements more or less continuously, or in multiple points of time—to increase the chances of detecting IOP diurnal variations which may not be adequately captured and evaluated when a patient has to visit a medical office for an IOP measurement.

There may be provided a tonometer that may be configured to measure, store and transmit IOP readings remotely, possibly several times a day, without a visit to the doctor's office, while at the same time providing information to the patient.

There may be provided a tonometer that can be easily transported and used at subject's home.

There may be provided a tonometer that may be easy to use both by trained personnel and subjects themselves.

There may be provided a tonometer that may be operated in any position and be usable with pediatric and geriatric patients and other ‘difficult’ subjects, like those encountered in veterinary practice, without anesthesia or constraints.

There may be provided a tonometer that does not require any calibration, or normalization using a measured value from a second “gold standard” device.

There may be provided a tonometer that, additionally to measuring IOP, also captures, stores and uploads to a remote data center high definition images or video or the users' eye, possibly under selectable lighting conditions.

The tonometer operation may be essentially automatic and may start upon subject's or operator's turning it on. The tonometer may have an internal compressed air generator, either in the form of a manual pump (e.g., bulb), an electrical pump similar to the ones found in portable blood pressure monitors, or a spring-loaded or electrically actuated piston moving within a cylinder.

The tonometer may include a sealed mask or goggles applied on the face, covering the eye and some areas of skin around the eyes. While the compressed air source is active, it increases the air pressure over the eyes to some predetermined level, and then releases it slowly—very much like what is does in standard home blood pressure monitors. Other pressure vs. time profiles are possible, as dictated by the measuring algorithm.

The tonometer may include a target on which the subject may stare.

The tonometer may include an illumination apparatus that illuminates the eye(s) with light of different wavelengths or white light to enable quality image capture, and possibly enhance blood vessels contrast under green or violet wavelengths.

A video camera captures images of some or all visible parts of the eye, as well as some area around the eye, while these pressure changes are in play, and an image analysis process is used to identify blood vessels, or areas rich in vessels both over or around the eye (outside the IOP pressure field) and inside the eye (exposed to IOP).

At different air pressures veins in the camera field of view will start pulsating and totally collapsing at higher air pressures. At even higher pressures arteries will start pulsating and collapsing. The vessels inside the eye are exposed to the air pressure inside the mask, plus the IOP, while vessels outside the eye are only exposed to the air pressure, but not to the IOP. By comparing (or extracting any other relationship between) the pressures at which blood vessels outside the eye pulsate or collapse to the pressure at which vessels inside the eye pulsate or collapse, it is possible to calculate the IOP.

### BRIEF DESCRIP HON OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates an example of a tonometer block diagram.

FIG. 2 illustrates an illustration of an eye with key areas marked.

FIG. 3 illustrates an example of a simulation of the reading of the Anterior Ciliary Artery diameter pulsations.

FIG. 4 is an example of an adjustable pressure mask.

FIGS. 5A-C are vessels pulsation amplitude heatmaps of an eye under several external pressures.

FIGS. 6A-B are graphs derived from eye heatmaps showing sclera (dotted line) and iris (solid line) pulsation amplitudes over an external pressure range applied to the right eye (FIG. 6A) and left eye (FIG. 6B).

FIG. 7 is a flowchart illustrating image-processing to derive IOP according to the teachings of the present invention.

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Any reference in the specification to a system should be applied mutatis mutandis to a method that can be executed by the system.

Because the illustrated at least one embodiment of the present invention may for the most part, be implemented using micro-electro-mechanical system (MEMS) components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method.

Devices for measuring intra-ocular pressure are well known in the art. Such devices typically measure a force required to generate a defined deformation of the cornea and calculate the IOP based on such force measurement. Such a force can be applied directly to the cornea or through a pulse of air. Although clinically-used tonometers can provide reliable results there remains a need for a portable tonometry device that provides reliable IOP readings in a non-clinical setting.

While reducing the present invention to practice, the present inventors have devised an approach that can be used to measure IOP of both eye simultaneously without directly contacting the cornea. The present approach can be used in a non-clinical setting by a non-skilled. individual.

Thus, according to one aspect of the present invention there is provided a system for determining an IOP of a subject (also referred to herein as a tonometer).

The system includes a pressurizing device for applying pressure of varying magnitude over an external surface of an eye of the subject and a monitoring device for monitoring internal vasculature of the eve and vasculature on or around the eve.

The pressurizing device can include a cup-shaped element for sealingly covering the eye and a pressure-generating mechanism such as a manually-operated (e.g., bulb, bellows) or electrical pump (e.g., peristaltic pump) for pressurizing a space formed over the eye by the cup-shaped element. The cup can form a part of a goggle or mask with both cups of the goggle/mask being simultaneously operated to provide IOP readings. The pressure in the cup can be a gradually increasing/decreasing pressure over a range of 0-120 mmHg. Alternatively, and in order to speed the measuring process, the fluid (e.g., air) pressure applied to the eye can be scanned through discrete values where a peak of pulsation/collapse is expected. If such a peak is not detected the scan can resume at different values until the peak is identified.

The pressurizing device can alternatively be a pad configured for applying a controlled pressure over one or bath eyelids when the eye or eyes are open.

The vasculature in and on/around the eye(s) is monitored by any modality capable of identifying pulsation or collapse of blood vessels. Examples include a visible light color camera, a BW camera, an infrared or UV video camera, an ultrasound distance or Doppler transducer or an opto-reflective distance sensor.

Vasculature in the eye (also referred to herein as “internal blood vessels”) refers to any blood vessels that are subjected to a combination of intra-ocular pressure and atmospheric pressure. Vasculature on/around the eye (also referred to herein as “external blood vessels”) refers to any blood vessels subjected to atmospheric pressure only.

Examples of internal blood vessels include, but are not limited to, the major and minor arterial circles of the iris and any vessels that reside on or in the iris, portions of the anterior ciliary arteries from the point beyond passing through the sclera and into the eye, and arteries visible on the retina such as the retinal artery, and any other vessels that normally experience IOP.

Examples of external blood vessels include, but are not limited to, the anterior ciliary arteries segments after exiting from the rectus muscles and positioned externally on the sclera.

The internal blood vessels can be monitored at the iris of the eye whereas the external blood vessels can be monitored on the sclera of an eye. Alternatively, monitoring of internal vessels can be through pupil on the retina, while external vessels can be monitored at the internal surfaces of the eyelids or the medial canthus.

The present system further includes a processing unit for correlating a first pressure or pressure range leading to pulsation or collapse (decrease in vessel size) of the internal vasculature of the eye and a second pressure or pressure range leading pulsation or collapse of the vasculature on or around the eye to thereby derive an IOP of the subject. Peak pulsation can be monitored by detecting cyclic image changes at the heart rate while partial or total collapse of blood vessels can be monitored by average or relative change in color (e.g., shift from red to green-blue).

The measurement rational assumes that since all said blood vessels are fed from one major vessel, the ophthalmic artery, the internal pressure in all these vessels is identical—especially in no-flow situations that exist when the vessels are restricted by external pressure. The internal vessels experience a surrounding pressure that is the sum of the IOP and the air pressure over the eye, while the external vessels only experience the air pressure over the eye. Since both vessels groups start pulsating and collapse when external pressure increases over the internal blood pressure, the difference between the pressure or pressure range leading to pulsating of internal and external vessels is equal to the IOP. It may be possible to correlate the pressure at collapse of sclera arteries with systemic blood pressure (BP), to thereby eliminate the need to measure the pressure of sclera vessel collapse.

The Examples section hereinbelow provides a detailed description of how the pressure or pressure range of internal and external blood vessels is measured at peak pulsation and how IOP is derived from these measurements.

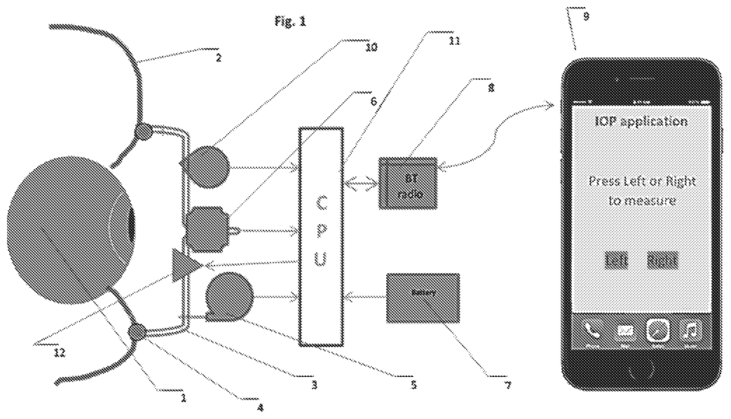

Referring now to the drawings, FIG. 1 illustrates one embodiment of the present system which is also referred to herein as a tonometer.

In FIG. 1 one or both eyes 1 in the skin around the eyes 2 are covered with an airtight mask 3. The mask is sealed around the edges where it is resting against the skin with a skin-compatible seal 4. The mask may comprise two separate volumes—one over each eye, or one volume over both eyes. The air pressure inside the volume defined by the face and the mask is controlled by air pressure pump 5 operable through commands from the CPU 11. The pump can be a membrane, piston, peristaltic or any other type of pump fed from room air or other gas source. Alternatively, it can be implemented as a pressurized container with a controlled valve to release a pressurized gas into the mask volume. The pressure inside the mask volume is measured by pressure sensor 10 in fluid communication with the internal volume of the mask, and can be in the range 0 mmHg to 120 mmHg. Readings from the pressure sensor are sent to the CPU 11 and are used to control the static pressure as well as any pressure changes inside the mask. The mask can be made opaque to minimize interference to the measuring process from external illumination, if desired. Naturally, the mask seal should withstand such pressures without a significant leak, or the pump is designed to compensate for some leaks as needed for users with different facial shapes and sizes. The mask itself can be made adjustable with varying widths, inter-pupil distances, heights, nasal recess, temple covers and more. The strap holding the mask to the face should be made adjustable to fit heads of different sizes, but it may not be stretchable because the air pressure inside pushes the mask away from the face, and the strap should resist this force which may reach 10 Kg or more at the higher pressures. The strap should have a fast release buckle to allow easy wearing and removal of the mask once size and configuration were set without having to readjust the size.

In front of each eye, a video camera 6 is installed. A dual device may have two cameras with adjustable locations so that they be moved to be in front of each eye, or a device can have camera bracket where a single camera is moved from one eye to the other to save cost. The video stream or a series of still images from the camera is sent to the CPU 11 for initial processing. The video camera focal distance is adjusted to capture a detailed image of the eye and its surrounding skin, eyelids and other anatomical features around the eye. The camera field of view may include the iris, cornea, sclera, pupil and any other part of the front of the eye, as well as the eyelids and skin around the eye. Additionally, in some embodiments, the camera is also equipped to provide video streams of the retina and other structures inside and at the back of the eye.

Illumination of the eyes inside the mask volume is provided by one or more light sources 12 at several different colors, which may include all visible colors as well as near IR in the 700-1200 nm range, or near UV in the 300-400 nm range. Light sources 12 can be fluorescent, incandescent or LEDs. One or more of the LEDs may be operated at any given time as needed by the measuring process, and the intensity of each active LED is also changeable under CPU 11 control. The LEDs may be all concentrated in a single location in the mask so as to illuminate both eyes at the same time, or distributed on the mask internal walls or in the mask volume as desired. More than one LED may be used for each color. LEDs illumination may be synchronized with the cameras capture timing to save energy and get better illumination without bothering the user with too bright light.

The same or additional LEDS can provide high intensity ambient light to make the pupil contract, thus exposing more of the iris for view and image capturing. Polarizers in front of the LEDs and/or camera may help reduce reflections and glare from the wet eye surface.

The camera, pressure pump and pressure sensor are all controlled by CPU 11 that runs the test routine, and all are powered by the battery 7 which can be primary or rechargeable, or by a wall power supply . The CPU 11 runs the test routine that completes the measurement, or can sends raw or semi-processed data as images and other data to a smartphone or other external computational device 9 for further processing and calculating the IOP, as well as managing the measurement, storing the data from later use, or uploading the data or measurement results to the cloud or a remote server for safe storage, remote monitoring the results, big data analysis over the results from many patients and so on.

It should be understood that the above description is exemplary only, and should not be viewed as the unique embodiment of the invention.

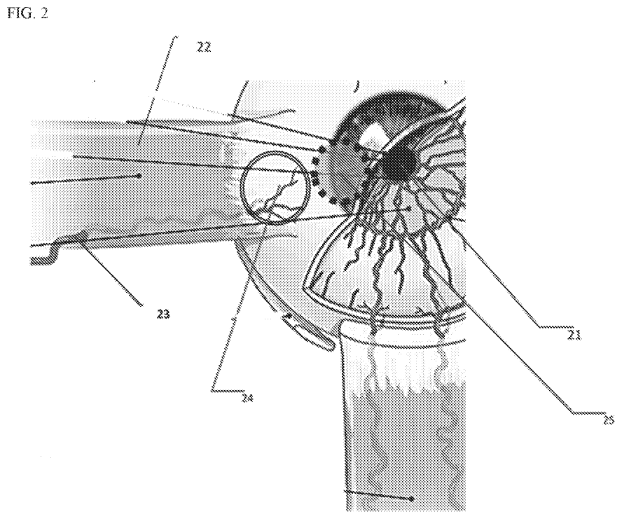

In FIG. 2 the patients' eye 21 is shown in close-up, with all its front-viewable arteries. On the left, the lateral rectus muscle 22 passes the Anterior Ciliary Artery 23 which exits the muscle and travels a short distance over the sclera in the area marked with the double-line circle 24 on the outside of the eye under the conjunctiva, where it is only exposed to the air pressure present over the eye. It then passes through a well in the sclera to join other ciliary arteries from other rectus muscles in the major circle of the iris. While passing in this area, marked with a dotted/shaded circle 25, it is exposed to the pressure inside the eye, which is the pressure of the air over the eye plus the pressure generated by the fluids of the eye—the IOP.

The image of the eye, as shown in FIG. 2 is captured by the camera 6 for each eye, and fed for analysis to the CPU 11. The image can be either analyzed by the onboard CPU, or it can be sent for processing in real time or in post processing in the remote connected device 9. The image analysis and IOP calculation software performs the algorithm described in the Examples section which follows. Naturally, the software—either in the on board CPU or in the remote device, may elect to turn on one or more of the LED to get the best video images of the arteries or other vessels for analysis. Of special note is illumination at 480-520 nm, considered optimal for blood vessels visualization.

The video of the eye and the analysis of the images are preformed while the pressure inside the mask volume is changed over a range which is a partial range of the total 0 mmHg to some maximum determined by the software as the pressure at which the arteries totally collapse, which may be as high as 120 mmHg. While this pressure is changing, the measuring process runs the algorithm to determine the IOP.

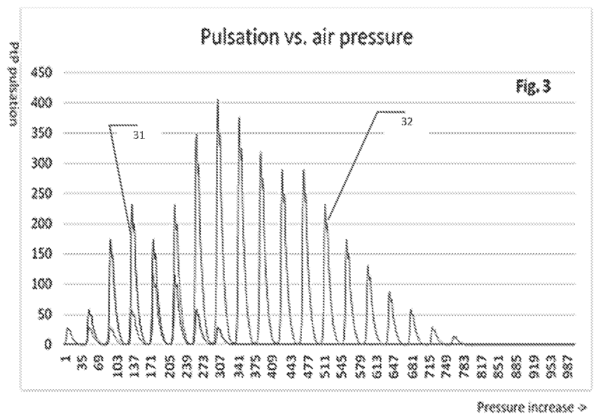

FIG. 3 is a simulation of the reading of the Anterior Ciliary Artery 23 diameter change due to pulsations, as derived from the video image of the front of the eye as the air pressure over the eye is changing over the range including the blood pressure inside the artery. It should be noted that other arteries may be monitored in order to determine the IPO.

The pulsating left peak line 31 in FIG. 3 represent pulsations of the part of the artery that is inside the eye, right after entering through a well in the sclera., or after it joins as part of the major circle of the iris. This is measured in the area inside the dotted/shaded circle 25 in FIG. 2. Note that the artery begins pulsating as the external pressure it experiences increases over the diastolic blood pressure inside the artery, increases to a maximum amplitude, and drops to a minimum value as the external pressure becomes greater than the systolic blood pressure inside the artery.

The same applies for the pulsating right peak line 32 in FIG. 3. representing the pulsations of the same artery, in the section of its travel outside the eye, after it emerged from the muscle, and before it entered the eye volume, represented by the area in the double-line circle 24 in figure z.

Both traces represent pulsations of the same artery, and because the distance between the two areas is just 1-3 mm, both segments have the same blood pressure inside, and similar wall properties. The difference in response of the artery sections to the air pressure over the eye is because the part inside the eye experiences the external air pressure PLUS the IOP, while the part that is outside the eye only experiences the air pressure.

The IOP can therefore be deduced from the shift in pressure response between the two graphs. This shift can be calculated by looking at the points where pulsations begin, end, reach a maximum or by using any other method. The preferred embodiment for performing this calculation is to calculate cross correlation between the two graphs envelopes at different pressure shifts, and find the pressure shift that produces the highest cross-correlation, which is the desired IOP.

Once IOP is determined, the same process of increasing air pressure over the eye and looking at pulsations of specific vessels in the eye can be repeated or data from these additional vessels taken at the same time as the initial cycle, in order to measure other important parameters. In example, looking at the central retinal vein response under pressure allows the measurement of blood pressure in that artery, which is equal to the intra-cranial pressure, and the same measurement on the central retinal artery can also be used to estimate patency of the carotid arteries—similarly as is currently done with ophthalmodynamometery testing but in a non-contact option, and with IOP compensation.

Since the pulsations are generated by blood pressure changes due to the heart activity, heart rate readings are also possible at the same time. Heart rate variability and arrhythmias may also be detected from the video images of the pulsating vessels.

With the vessels exposed in plain view of the camera, it is possible to measure oxygen saturation separately in the arteries and in the veins which are in the camera field of view. This can be accomplished using standard multiple wavelengths, reflective oximetry techniques. This may apply to vessels which are seen separately in the eyes, as well as to standard reflective oximetry from areas rich in blood vasculature around the eye such as the lacrimal caruncle. Small areas may be isolated for measurement in the video stream from the camera under illumination at different wavelengths such as the red and IR wavelengths.

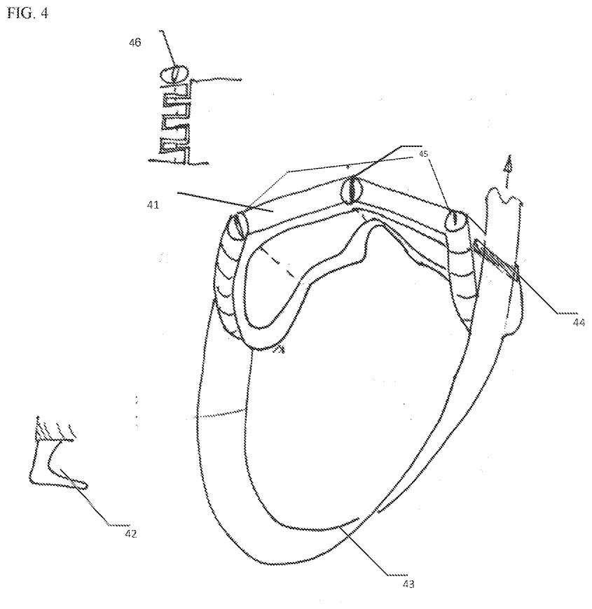

FIG. 4 is an example of an adjustable pressure mask. The mask body 41 is made of a very rigid plastic, which can withstand the forces of several Kg which will develop due to the air pressure inside. The mask frame is divided in four parts connected with three rotatable joints 45—one at the center, and two near the left and right ends. Adjusting the joints allows the fitting of the mask to the user's face, and then the joints are locked in that position by turning a finger screws 46. The front of the mask in front of the eyes is transparent so the optics can capture images of the eyes. One or more cameras and illuminators are adjustably mounted on the front of the mask so the eyes are in their field of view.

The frame edges towards the skin are covered with a very soft seal 42 that forms a closed volume under the mask, which keeps the air pressure inside. The seal is a pneumatic seal with an L shaped cross-section with the leaf entering under the frame and may be protruding over the skin inside the mask volume.

The mask is held on the face with a soft, elastic but none non-stretchable band 43 that can be adjusted using the clasp 44 which the user pulls until the mask is tight-fitting on his face.

Although the previous text referred to a camera is should be noted that other sensors may be used for sensing the pulsation amplitude as a function of external pressure. For example—a depth sensor other than a camera may be used to sense the pulsation of the arteries.

The tonometer may be configured to measure temporal variation of average (over area) intensity, contrast, motion or any other parameter of the image at a specific color or color range, of specific areas of the image at the heart rate and phase (derived from the ECG signal).

Since the pulsating arteries change their dimensions, it may be possible to detect these changes remotely using ultrasonic (distance or Doppler) sensors, optical distance sensors (using reflection of a light beam off the eye) or any other sensor that is capable of detecting the micrometer range motions at the heart rate such as laser interferometric sensors This may be especially relevant for detecting the pulsations of the arteries inside the eye, being partially or totally hidden by the corneal colored structures.

Any of the figures may or may not be in scale.

Any reference to any of the terms “comprise”, “comprises”, “comprising” “including”, “may include” and “includes” may be applied to any of the terms “consists”, “consisting”, “and consisting essentially of”.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front, ” “back, ” “top, ” “bottom, ” “over, ” “under ” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Those skilled in the art will recognize that the boundaries between elements are merely illustrative and that alternative embodiments may merge elements or impose an alternate decomposition of functionality upon various elements. Thus, it is to be understood that the architectures depicted herein are merely exemplary, and that in fact many other architectures can be implemented which achieve the same functionality.

Any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations are merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single device. Alternatively, the examples may be implemented as any number of separate devices or separate devices interconnected with each other in a suitable manner. However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one ” and “one or more ” in the claims should not be construed to imply that the introduction of another claim element by the indefinite articles “a” or “an” limits any particular claim containing such introduced claim element to inventions containing only one such element, even when the same claim includes the introductory phrases “one or more ” or “at least one ” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements.

While certain features of the invention have been illustrated and described herein, many modifications, substitutions, changes, and equivalents will now occur to those of ordinary skill in the art. it is, therefore, to be understood that the appended claims are intended to cover all such modifications and changes as fall within the true spirit of the invention.

Additional objects, advantages, and novel features of the present invention will become apparent to one ordinarily skilled in the art upon examination of the following examples, which are not intended to be limiting.

### EXAMPLES

Reference is now made to the following example, which together with the above descriptions, illustrate the invention in a non-limiting fashion.

### IOP Measurement

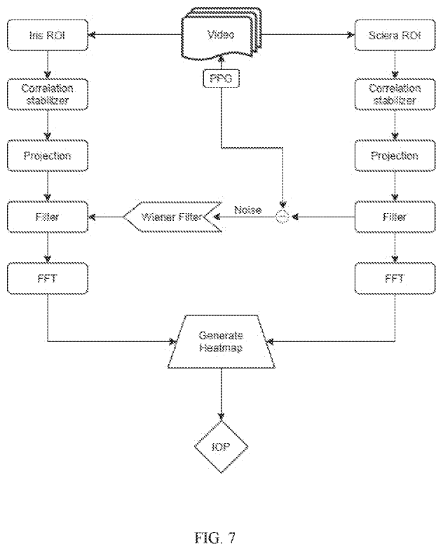

The present approach was tested by applying pressure over an eye and calculating the difference between the pressure that leads to the highest pulsation amplitude of vessels on the sclera (external vessels), and the pressure that leads to the highest pulsation amplitude of vessels in the iris (internal vessels); FIG. 7 outlines the present process.

### Eye Monitoring

An 18MP, 2/3″ sensor camera (IDS) was used to capture a color video stream at 50 frames per second, and 2Mpixel resolution (1920×1080) of an eye. The camera was equipped with a 2/3″. 1.8/25 mm C-mount lens (Kowa) lens providing a 10 μm/pixel resolution. The eye was illuminated by a white LED matrix. Video was sent to a PC for processing.

### Eye Pressurizing

A face mask having an elastomeric pneumatic seal was used to cover both eyes to form a sealed volume over the eyes. The air pressure inside the mask was generated and controlled by a DC motor membrane pump with an Arduino controller and running at a 10 Hz main loop. Pressure readings from a pressure sensor mounted in the mask were serially sent to the Arduino controlling the process as well as the PC collecting all the data.

### Image Analysis and IOP Calculation

Video analysis software was used to extract a clean “pulsation” signal from specific areas on the sclera and iris that are found to present a pulsating behavior under specific ambient pressure levels. The software analyzes the video in view of 10 Hz pressure data received from the pressure sensor.

The pupil was detected in each frame using a mask search, and its location was used as the main reference point for locations of other features or selected areas on the eye.

Briefly, each frame of the video was divided in to sub-areas of 20×20 pixels each. Each sub-area was tracked to the next frame by calculating the cross correlation of the sub-area with a 20×20 sub-area in the new frame, and searching for the maximal cross-correlation value by sweeping its location by a single pixel over a 50×50 pixel search area. A total of 900 values were calculated, and the new sub-area location was set to the location of maximal cross-correlation. This process was repeated for all possible sub-areas in the image, and over all of the frames of the captured video. This process was necessary since vessels in the eye reside on several layers that move with respect to each other.

Following stabilization, multiple vectors were generated with each vector representing consecutive values of a specific pixel across all frames in the video.

Large deviations from average values that indicate outliers in the measurements, such a blinks, user motions or video artifacts were removed by replacing these values with the last valid value. Each of the single pixel vectors generated were projected to a different color space. The new space was created by first generating three new vectors for the green, red and Value (in INV space), using a PCA operator (in SVD on these vectors. The vector representing the color which is least present in the original vector was then selected. All pixels in the original vector were projected on this vector and then smoothed and high-passed. This process greatly increased the SNR of the pulsation signal vs. noise and artifacts.

Since the user's heart rate may shift while the test is ongoing, the energy is calculated over a range of frequencies around the original heart rate (HR). A Fourier transform of the resulting vectors was carried out in order to calculate the energy at HR frequency.

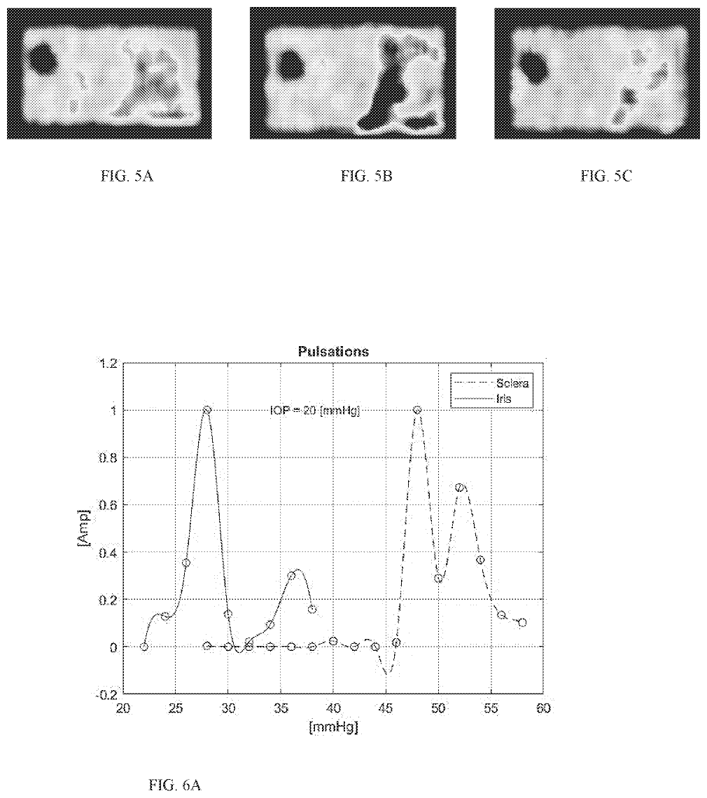

A heatmap was generated for each sub-area and the signal was depicted as a colored square on the eye image (FIGS. 5A-C). Larger energies are darker, while lower energies are lighter; the dark dot on the top left is the pupil.

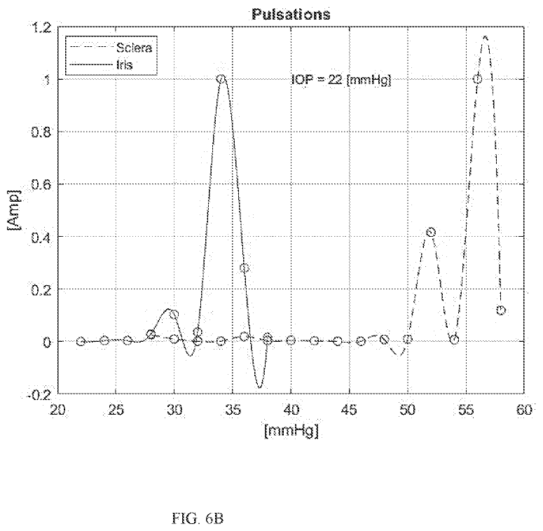

The IOP was then calculated from the heat maps. The sub-areas that demonstrated the larger variation of pulsation energy as a function of ambient air pressure were selected, and their data was averaged and plotted as a function of pressure (FIGS. 6A-B) for both scleral and iris vessels. The difference between the scleral maximal energy pressure and the iris maximal energy pressure is the IOP.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for determining an IOP of a subject comprising:
(a) a pressurizing device for applying pressure of varying magnitude over an external surface of an eye of the subject;
(b) a monitoring device for monitoring internal vasculature of said eye and vasculature on or around said eye; and
(c) a processing unit for correlating a first pressure or pressure range with pulsation or collapse of said internal vasculature of said eye and a second pressure or pressure range with pulsation or collapse of said vasculature on or around said eye to thereby derive the IOP of the subject.

2. The system of claim 1, wherein a value of said IOP is derived by calculating a shift between said first pressure range and said second pressure range.

3. The system of claim 1, wherein said pressurizing device includes a cup-shaped element for sealingly covering said eye and a pressure-generating mechanism for pressurizing a space formed over said eye by said cup-shaped element.

4. The system of claim 3, wherein said cup forms a part of a goggle or mask.

5. The system of claim 3, wherein said pressure-generating mechanism is a pump.

6. The system of claim 1, wherein said monitoring device is a visible light camera, a non-visible light camera or an ultrasound transducer.

7. The system of claim 1, wherein said internal vasculature is monitored on an iris of said eye.

8. The system of claim 1, wherein said vasculature on or around said eye is monitored at a sclera of said eye.

9. The system of claim 4, wherein said monitoring device is attached to said goggle or mask.

10. The system of claim 1, wherein said processing unit add a pressure offset to said first pressure range.

11. The system of claim 1, wherein said pressurizing device includes a pad for applying a controlled pressure over one or both eyelids.

12. The system of claim 1, wherein said pressure of varying magnitude is a gradually increasing pressure

13. A method of determining an IOP of a subject comprising:
(a) applying pressure of varying magnitude over an external surface of an eye of the subject;
(b) monitoring internal vasculature of said eye and external vasculature on or around said eye; and
(c) correlating a first pressure or pressure range with pulsation or collapse of said internal vasculature of said eye and a second pressure or pressure range with pulsation or collapse of said external vasculature on or around said eye to thereby derive the IOP of the subject.

14. The method of claim 13, wherein a value of said IOP is derived by calculating a shift between said first pressure range and said second pressure range.

15. The method of claim 13, wherein said internal vasculature is monitored on an iris of said eye.

16. The method of claim 13, wherein said vasculature on or around said eye is monitored at a sclera of said eye.

17. The method of claim 13, wherein a pressure offset is added to said first pressure range.

18. The method of claim 13, wherein said pressure of varying magnitude is a gradually increasing pressure.
